(19) United States
(12) Patent Application Publication
(10) Pub. No.: US 2026/0253745 A1
(43) Pub. Date: Aug. 27, 2026

(54) METHOD, DEVICE, AND SYSTEM FOR REAL-TIME VIRUS INFECTION RISK MONITORING

(71) Applicant: Tien-Tse HSIEH, Tainan City (TW)

(72) Inventor: Tien-Tse HSIEH, Tainan City (TW)

(21) Appl. No.: 19/651,831

(22) Filed: Apr. 20, 2026

Related U.S. Application Data
(63) Continuation-in-part of application No. 18/465,974, filed on Sep. 12, 2023.
(60) Provisional application No. 63/405,929, filed on Sep. 13, 2022.

(51) Int. Cl. G16H 50/30; G05B 15/02; G05D 22/02; G05D 23/19; G05D 27/02

(52) U.S. Cl. CPC G16H 50/30; G05B 15/02; G05D 22/02; G05D 23/19; G05D 27/02

(57) ABSTRACT

A method for real-time virus infection risk monitoring is provided. A CO₂ sensor, a particulate matter sensor, a temperature sensor, and a humidity sensor are utilized to respectively detect an ambient CO₂ concentration, an ambient particulate matter concentration, an ambient temperature, and an ambient humidity in a first environment. A first difference between an ambient CO₂ concentration and a reference CO₂ concentration and a second difference between an ambient particulate matter concentration and a reference particulate matter concentration are computed by a processor. The first environment is determined to have a high infection risk of a specific virus by the processor in response to a condition being met.

### RELATED APPLICATIONS

This application is a continuation-in-part application of the U.S. application Ser. No. 18/465,974 filed Sep. 12, 2023, which claims priority to U.S. Provisional Application No. 63/405,929, filed Sep. 13, 2022, all of which are herein incorporated by reference.

### BACKGROUND

### Technical Field

The present disclosure relates to virus infection risk monitoring, and more particularly to a method, a device, and a system for real-time virus infection risk monitoring.

### Description of Related Art

Conventional assessment techniques of virus infection risks have many problems in practical applications. The problems may influence the efficiency and convenience of the assessment techniques.

First, these assessment techniques often require a lot of time. For example, fluorescence analysis is used for detection. This is time-consuming and cumbersome. These assessment techniques also require a large amount of consumables, such as chemicals or filters, which not only increase costs but also place an unnecessary burden on the environment. Another disadvantage of the conventional assessment techniques is that the cost of the equipment is high. Many assessment techniques require expensive equipment and devices, which may be a burden for many laboratories and institutions.

The conventional assessment techniques are difficult to achieve consistent results quickly when performing virus risk detections in different locations. They cannot immediately alert the public to take corresponding protective measures, so it is impossible to reduce the risk of infection effectively. The conventional assessment techniques have many shortcomings. There is a need for a new technique to overcome these problems and improve the efficiency and reliability of detection.

### SUMMARY

In view of the foregoing deficiencies of the existing technology, the technical problem to be solved by the present disclosure is to provide a method, a device, and a system for real-time virus infection risk monitoring.

According to one aspect of the present disclosure, a method for real-time virus infection risk monitoring is provided. A CO₂ sensor, a particulate matter sensor, a temperature sensor, and a humidity sensor are utilized to respectively detect an ambient CO₂ concentration, an ambient particulate matter concentration, an ambient temperature, and an ambient humidity in a first environment. A first difference between the ambient CO₂ concentration and a reference CO₂ concentration and a second difference between the ambient particulate matter concentration and a particulate matter concentration are computed. The first environment is determined to have a high infection risk of a specific virus by the processor in response to the ambient temperature being in a temperature range for transmission of the specific virus, the ambient humidity being in a humidity range for transmission of the specific virus, and one of the following conditions being met: the first difference is greater than a first CO₂ concentration difference threshold, and the second difference is greater than a first particulate matter concentration difference threshold; the first difference is greater than the first CO₂ concentration difference threshold, and the second difference is greater than a second particulate matter concentration difference threshold and less than or equal to the first particulate matter concentration difference threshold; or the first difference is greater than a second CO₂ concentration difference threshold and less than or equal to the first CO₂ concentration difference threshold, and the second difference is greater than the first particulate matter concentration difference threshold.

In some embodiments, the method further includes controlling an electronic output component to output an indication for an infection risk warning of the specific virus by the processor in response to determining that the first environment has the high infection risk of the specific virus.

In some embodiments, controlling the electronic output component to output the indication is controlling a display to show a health alert message for the infection risk warning of the specific virus.

In some embodiments, the method further includes sending a command from the processor to a heating, ventilation, and air conditioning (HVAC) system in the first environment to work for adjusting the ambient CO₂ concentration, the ambient particulate matter concentration, the ambient temperature, or the ambient humidity.

In some embodiments, the ambient temperature is less than 30° C., the ambient humidity is less than 45% or greater than 90%, and the specific virus is coronavirus.

In some embodiments, the ambient temperature is less than 22° C., the ambient humidity is less than 50%, and the specific virus is coronavirus.

In some embodiments, the reference particulate matter concentration and the ambient particulate matter concentration are PM2.5 concentrations or PM10 concentrations.

In some embodiments, the first CO₂ concentration difference threshold is 1200 ppm, the second CO₂ concentration difference threshold is 400 ppm, the first particulate matter concentration difference threshold is 40 μg/m³, and the second particulate matter concentration difference threshold is 20 μg/m³.

In some embodiments, the reference CO₂ concentration and the reference particulate matter concentration are provided from a local database or a cloud database.

In some embodiments, the reference CO₂ concentration and the reference PM2.5 concentration are detected in a second environment different from the first environment and respectively by the CO₂ sensor and the particulate matter sensor.

In some embodiments, the first environment and the second environment correspond to an identical space and different detection times.

In some embodiments, the first environment corresponds to an indoor space, and the second environment corresponds to an outdoor space neighboring the indoor space.

In some embodiments, the method further includes sending an indication with a location of the first environment from the processor to an external device for an infection risk warning of the specific virus for the first environment after determining that the first environment has a high infection risk of the specific virus.

In some embodiments, the method further includes adjusting the reference CO₂ concentration, the reference particulate matter concentration, the temperature range, and the humidity range of the specific virus by the processor according to current virus of disease information.

According to another aspect of the present disclosure, a device for real-time virus infection risk monitoring is provided. The device includes a CO₂ sensor, a particulate matter sensor, a temperature sensor, a humidity sensor, and a processor. The CO₂ sensor is configured to detect an ambient CO₂ concentration in an environment. The particulate matter sensor is configured to detect an ambient particulate matter concentration in the environment. The temperature sensor is configured to detect an ambient temperature in the environment. The humidity sensor is configured to detect an ambient humidity in the environment. The processor is configured to compute a first difference between the ambient CO₂ concentration and a reference CO₂ concentration, compute a second difference between the ambient particulate matter concentration and a reference particulate matter concentration, and determine that the environment has a high infection risk of a specific virus in response to the ambient temperature being in a temperature range for transmission of the specific virus, the ambient humidity being in a humidity range for transmission of the specific virus, and one of the following conditions being met: the first difference is greater than a first CO₂ concentration difference threshold, and the second difference is greater than a first particulate matter concentration difference threshold; the first difference is greater than the first CO₂ concentration difference threshold, and the second difference is greater than a second particulate matter concentration difference threshold and less than or equal to the first particulate matter concentration difference threshold; or the first difference is greater than a second CO₂ concentration difference threshold and less than or equal to the first CO₂ concentration difference threshold, and the second difference is greater than the first particulate matter concentration difference threshold.

In some embodiments, the device further includes an electronic output component that is configured to output an indication for an infection risk warning of the specific virus in response to the environment being determined to have the high infection risk of the specific virus.

In some embodiments, the processor is further configured to send an indication with a location of the environment to an external device through a wireless transceiver for an infection risk warning of the specific virus for the environment after the environment is determined to have the high infection risk of the specific virus.

In some embodiments, the processor is further configured to send an indication with a location of the environment to an external device for an infection risk warning of the specific virus for the environment after determining that the environment has the high infection risk of the specific virus.

In some embodiments, the processor is further configured to adjust the reference CO₂ concentration, the reference particulate matter concentration, the temperature range, and the humidity range of the specific virus according to current virus of disease information.

According to yet another aspect of the present disclosure a system for real-time virus infection risk monitoring is provided. The system includes an ambient detector and a server. The ambient detector includes a CO₂ sensor, a particulate matter detector, a temperature sensor, a humidity sensor, and a wireless transceiver. The CO₂ sensor configured to detect an ambient CO₂ concentration in an environment. The particulate matter sensor is configured to detect an ambient particulate matter concentration in the environment. The temperature sensor is configured to detect an ambient temperature in the environment. The humidity sensor is configured to detect an ambient humidity in the environment. The wireless transceiver configured to send the ambient CO₂ concentration, the ambient particulate matter concentration, the ambient temperature, and the ambient humidity. The server is communicatively connected to the ambient detector. The server is configured to receive the ambient CO₂ concentration, the ambient particulate matter concentration, the ambient temperature, and the ambient humidity from the ambient detector, compute a first difference between the ambient CO₂ concentration and a reference CO₂ concentration, compute a second difference between the ambient particulate matter concentration and a reference particulate matter concentration, and determine that the environment has a high infection risk of a specific virus if the ambient temperature is in a temperature range for transmission of the specific virus, the ambient humidity is in a humidity range for transmission of the specific virus, and one of the following conditions is met: the first difference is greater than a first CO₂ concentration difference threshold, and the second difference is greater than a first particulate matter concentration difference threshold; the first difference is greater than the first CO₂ concentration difference threshold, and the second difference is greater than a second particulate matter concentration difference threshold and less than or equal to the first particulate matter concentration difference threshold; or the first difference is greater than a second CO₂ concentration difference threshold and less than or equal to the first CO₂ concentration difference threshold, and the second difference is greater than the first particulate matter concentration difference threshold. The server further sends a report to the ambient detector to inform the ambient detector that the environment has the high infection risk of the specific virus.

The primary object of the present disclosure is to obtain an ambient CO₂ concentration, an ambient particulate matter concentration, an ambient temperature, and an ambient humidity in an environment through a device that can be portable and is easy to carry, and then determine the infection risk of a specific virus in the environment. The present disclosure has at least the advantages of easy portability, no consumables and real-time monitoring and warning.

The disclosure as well as a preferred mode of use, further objectives and advantages thereof will be best understood by reference to the following detailed description of illustrative embodiments when read in conjunction with the accompanying drawings.

### BRIEF DESCRIPTION OF THE DRAWINGS

The foregoing aspects and many of the accompanying advantages of this disclosure will become more readily appreciated as the same becomes better understood by reference to the following detailed description, when taken in conjunction with the accompanying drawings.

FIG. 1 is a schematic block diagram of a device for real-time virus infection risk monitoring in accordance with some embodiments of the present disclosure.

FIG. 2 is a flowchart of a method for real-time virus infection risk monitoring in accordance with some embodiments of the present disclosure.

FIG. 3 is a schematic diagram of a system for real-time virus infection risk monitoring in accordance with some embodiments of the present disclosure.

### DETAILED DESCRIPTION

For further illustrating the means and functions by which the present disclosure achieves the certain objectives, the following description, in conjunction with the accompanying drawings and preferred embodiments, is set forth as below to illustrate the implement, structure, features and effects of the subject matter of the present disclosure.

It will be understood that, although the terms “first,” “second,” and so on may be used herein to describe various elements, conditions and/or information, these elements, conditions and/or information should not be limited by these terms. These terms are only used to distinguish an element, condition and/or information from another element, condition and/or information.

The term “environment” herein is characterized by a specific space and a specific time. The specific space may be, for example, but not limited to, an open outdoor space or an enclosed or semi-open indoor space. The specific time may be the exact time of detection of ambient factors such as CO₂ concentration, PM2.5 particulate matter concentration, temperature, and/or humidity, or may be a predetermined time corresponding to reference ambient factors.

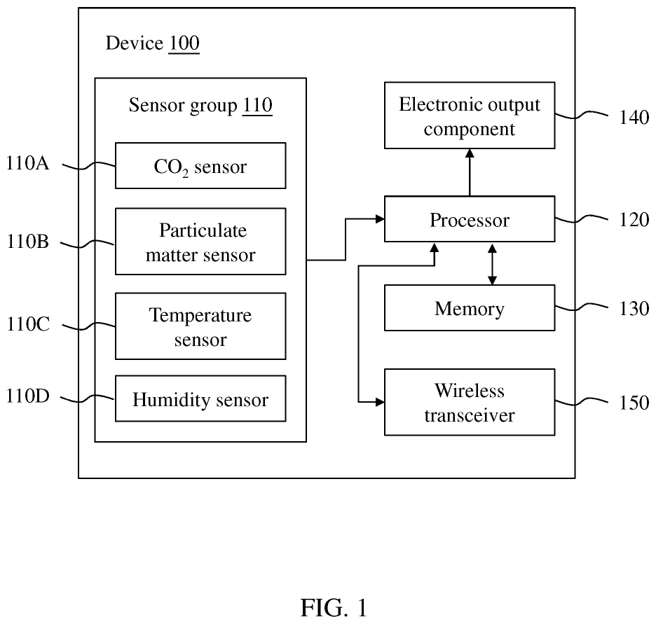

FIG. 1 is a schematic block diagram of a device 100 for real-time virus infection risk monitoring in accordance with some embodiments of the present disclosure. The device 100 may be a portable device for real-time monitoring the virus infection risk in an environment. In addition, the device may be powered by a battery (e.g., a lithium-ion battery) or by an external power source via, for example, a USB connection. As shown in FIG. 1, the device 100 includes a sensor group 110, a processor 120, a memory 130, an electronic output component 140, and a wireless transceiver 150.

The sensor group 110 includes a CO₂ sensor 110A, a particulate matter sensor 110B, a temperature sensor 110C, and a humidity sensor 110D, which are used to respectively detect an ambient CO₂ concentration, an ambient particulate matter concentration, an ambient temperature, and an ambient humidity in an environment. The particulate matter sensor 110B may be a PM2.5 sensor for detecting the concentration of particles that are 2.5 μm or less in diameter, a PM10 sensor for detecting the concentration of particles that are 10 μm or less in diameter, or another sensor suitable for detecting microparticles in the air. In some embodiments, two or more particulate matter sensors may be applied to particulate matter concentration detection. In a case in which a PM2.5 sensor and a PM10 sensor are applied, the PM2.5 sensor is used as the primary determination basis, and the PM10 sensor is used as the secondary determination basis. The PM2.5 concentration is the default of the ambient particulate matter concentration in general conditions. If the PM2.5 concentration is less than 5 μg/m³, the PM10 concentration may be used as the ambient particulate matter concentration in replace of the PM2.5 concentration. Each of the CO₂ sensor 110A, the particulate matter sensor 110B, the temperature sensor 110C, and the humidity sensor 110D may be implemented as a chip for portability of the device 100.

The processor 120 is electrically connected to the sensor group 110, and may be, for example, a central processing unit (CPU), a graphics processing unit (GPU), a microcontroller unit (MCU), a field-programmable grid array (FPGA), an application-specific integrated circuit (ASIC), or another circuit having computation and control functions. The processor 120 receives the detected ambient CO₂ concentration, ambient particulate matter concentration, ambient temperature, and/or ambient humidity from the sensor group 110, and determines the level of virus infection risk accordingly.

In particular, the processor 120 may compute a human activity factor of the environment from a reference CO₂ concentration, a reference particulate matter concentration, and the detected ambient CO₂ concentration and particulate matter concentration. The human activity factor is in proportion to the density of people in a specific space and/or the intensity of human activities. For example, a space with a large number of people is compared with a space with a small number of people, and the people in the space are moving or sitting still. The CO₂ concentration and particulate matter concentration in the former will be higher than those in the latter.

Then, the processor 120 may determine whether the environment has a high infection risk of a specific virus based on the ambient temperature, the ambient humidity, and the human activity factor. The survival rate and transmission speed of various viruses are affected by the ambient temperature and/or the ambient humidity, and different viruses have different peak periods. Thus, for a specific virus, the infection risk thereof can be determined based on the ambient temperature and the ambient humidity as well as the human activity factor.

In one example, the coronavirus (e.g., Covid-19) is highly transmissible (having a high infection risk) in a low humid and low temperature environment or in a high humid and low temperature environment. The transmissibility of the coronavirus is low when the ambient temperature is greater than 30° C. and the ambient humidity is between 45% and 60%. If the ambient temperature is less than 30° C., the ambient humidity is less than 45% or greater than 90%, and the human activity factor is high, the processor 120 determines that the environment has a high infection risk of Covid-19 and/or another coronavirus, and immediately generates an indication for an infection risk warning of Covid-19 and/or another coronavirus. On the contrary, if the ambient temperature is 38° C., the processor 120 determines that it is not easy to transmit Covid-19 in the environment and no warning of Covid-19 and/or another coronavirus is generated.

In another example, the influenza (e.g., influenza A virus) is highly transmissible (having a high infection risk) in a low temperature and dry environment. The transmissibility of the influenza is low when the ambient temperature is greater than 22° C. and the ambient humidity is greater than 50%. If the ambient temperature is less than 22° C., the ambient humidity is less than 50%, and the human activity factor is high, the processor 120 determines that the environment has a high infection risk of influenza, and immediately generates an indication for an infection risk warning of influenza.

The processor 120 may further send a command to a heating, ventilation, and air conditioning (HVAC) system in the environment to work for adjusting the ambient CO₂ concentration, the ambient particulate matter concentration, the ambient temperature, and/or the ambient humidity. The HVAC system may take appropriate actions to reduce the viral infection risk in the environment according to the command, such as, but not limited to, changing the air conditioner to a higher setting and/or decreasing the dehumidifier setting. The HVAC system may include an air purifier and/or an energy recovery ventilator (ERA), but is not limited thereto.

In addition, the processor 120 may further send an indication with a location of the environment to an external device (e.g., through the wireless transceiver 150) for an infection risk warning of a specific virus in the environment after determining that the environment has a high infection risk of the specific virus. In some embodiments, the processor 120 sends the indication to the mobile devices near the device 100, so that the nearby people may learn about the high infection risk of the specific virus in the environment and take appropriate precautions. The processor 120 may send the indication to a cloud server which stores information of locations with virus infection risk of the specific virus in its database. The cloud server may share the information of locations with high infection risk of the specific virus with the public, in order to prevent people from going to the locations.

The memory 130 is electrically connected to the processor 120. The memory 130 may store instructions that are performed by the processor 120 for performing real-time virus infection monitoring, and may store data such as the ambient CO₂ concentration, the particulate matter concentration, the ambient temperature, the ambient humidity, the monitoring result (e.g., whether or not the environment has a high infection risk of a specific virus) for each time for further analyses, and/or reference data (e.g., the reference CO₂ concentration, the reference particulate matter concentration, the temperature range, and the humidity range) that are pre-stored in a database thereof for virus infection risk determination by the processor 120. The reference data may be adjusted by the processor 120 according to the current virus of disease information from an external device (e.g., a server) for accurate and instant infection risk determination of a specific virus that is, for example, pandemic around the world or prevalent in an area where the device 100 is located.

The electronic output component 140 is connected to the processor 120. The electronic output component 140 may output an indication for an infection risk warning of a specific virus (may be controlled by the processor 120) if the processor 120 determines that the environment has a high infection risk of the specific virus. The electronic output component 140 may be a display, a light source (e.g., a light-emitting diode), a speaker, and so on, for outputting the indication by means of any one or a combination of two or more of warning lights, warning sounds or warning images. For example, if the electronic output component 140 is a display, it may show the indication as a health alert message for the infection risk warning of the specific virus.

The wireless transceiver 150 is electrically connected to the processor 120. The wireless transceiver 150 may be used for communicatively connect to a local device or a remote terminal (e.g., a mobile phone and/or a cloud server) through a wireless technology, such as cellular, Wi-Fi, Bluetooth, etc., for receiving data from the local device or the remote terminal and/or sending data to the local device or the remote terminal.

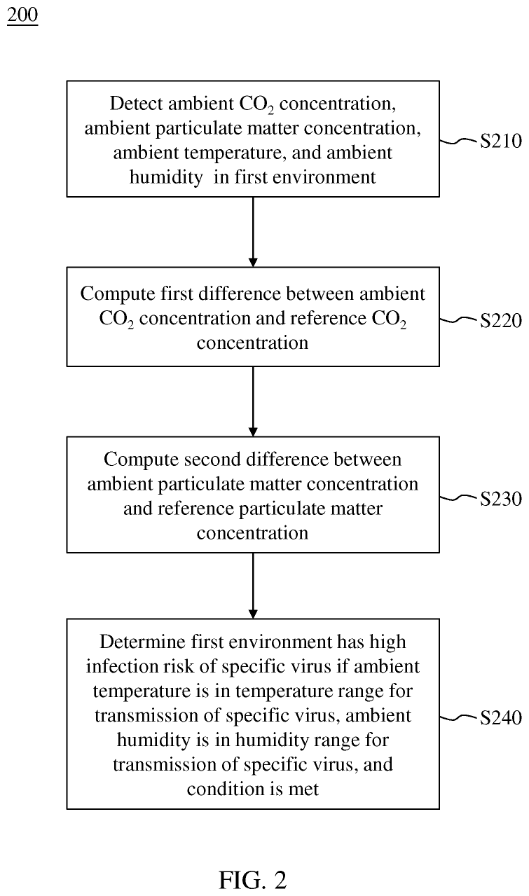

FIG. 2 is a flowchart of a method 200 for real-time virus infection risk monitoring in accordance with some embodiments of the present disclosure. The method 200 may be performed by a device having ambient detection and computation functions, such as the device 100 in FIG. 1. The steps of the method 200 are described as follows.

At Step S210, an ambient CO₂ concentration, an ambient particulate matter concentration, an ambient temperature, and an ambient humidity in a first environment are detected by utilizing a CO₂ sensor, and a particulate matter sensor (e.g., a PM2.5 sensor or a PM10 sensor), respectively.

At Step S220, a first difference between the ambient CO₂ concentration and a reference CO₂ concentration is computed by a processor.

At Step S230, a second difference between the ambient particulate matter concentration and a reference particulate matter concentration is computed by the processor.

The reference CO₂ concentration and the reference particulate matter concentration are reference values for comparison with the ambient CO₂ concentration and the ambient particulate matter concentration. These reference values may correspond to a second environment different from the first environment, and may be provided from a local database (e.g., in a built-in memory or through a local area network) or a cloud database (e.g., through the Internet). In some embodiments, the first environment and the second environment correspond to an identical space and different detection times. In some embodiments, the first environment corresponds to an indoor space, and the second environment corresponds to an outdoor space neighboring the indoor space.

Alternatively, the reference CO₂ concentration and the reference particulate matter concentration may be obtained by detection. That is, the reference CO₂ concentration and the reference particulate matter concentration may be detected in a second environment different from the first environment and respectively by the CO₂ sensor and the particulate matter sensor.

At Step S240, the first environment is determined to have a high infection risk of a specific virus by the processor if the ambient temperature is in a temperature range for transmission of the specific virus, the ambient humidity is in a humidity range for transmission of the specific virus, and a condition is met. The condition may be one of the following: the first difference is greater than a first CO₂ concentration difference threshold, and the second difference is greater than a first particulate matter concentration difference threshold; the first difference is greater than the first CO₂ concentration difference threshold, and the second difference is greater than a second particulate matter concentration difference threshold and less than or equal to the first particulate matter concentration difference threshold; or the first difference is greater than a second CO₂ concentration difference threshold and less than or equal to the first CO₂ concentration difference threshold, and the second difference is greater than the first particulate matter concentration difference threshold.

Specifically, the human activity which is defined as the density of people in a specific space and the intensity of human activities. The human activity factor is subject to the level of CO₂ impact and the level of particulate matter impact.

For the level of CO₂ impact, if the first difference is greater than the first CO₂ concentration difference threshold, the CO₂ impact is determined to be high; if the first difference is greater than the second CO₂ concentration difference threshold and less than or equal to the first CO₂ concentration difference threshold, the CO₂ impact is determined to be medium; if the first difference is less than or equal to the second CO₂ concentration difference threshold, the CO₂ impact is determined to be low. The high CO₂ impact implies that the number of people accommodated in the unit space is great, people are in close contact, and the chance of transmitting the virus via droplets, from the nose and mouth, is high. In some cases, the first CO₂ concentration difference threshold is 1200 ppm, and the second CO₂ concentration difference threshold is 400 ppm.

Similarly, for the level of particulate matter impact, if the second difference is greater than the first particulate matter concentration difference threshold, the particulate matter impact is determined to be high; if the second difference is greater than the second particulate matter concentration difference threshold and less than or equal to the first particulate matter concentration difference threshold, the particulate matter impact is determined to be medium; if the second difference is less than or equal to the second particulate matter concentration difference threshold, the particulate matter impact is determined to be low. The particulate matter impact implies that the concentration of particulate matter in the unit space is high. If the air in a space with a high particulate matter impact is not circulated, the amount of the virus suspended in the air increases, and the chance of transmitting the virus increases. In some cases, the first particulate matter concentration difference threshold is 40 μg/m³, and the second particulate matter concentration difference threshold is 20 μg/m³.

After the level of CO₂ impact and the level of particulate matter impact are determined, the human activity factor in the first environment is determined accordingly. The relationship between the human activity factor, the level of CO₂ impact, and the level of particulate matter impact is shown in Table 1.

TABLE 1

| | High CO₂ | Medium CO₂ | Low CO₂ |
|---|---|---|---|
| | impact | impact | impact |
| High particulate | High human | High human | Medium human |
| matter impact | activity factor | activity factor | activity factor |
| Medium particulate | High human | Medium human | Low human |
| matter impact | activity factor | activity factor | activity factor |
| Low particulate | Medium human | Low human | Low human |
| matter impact | activity factor | activity factor | activity factor |

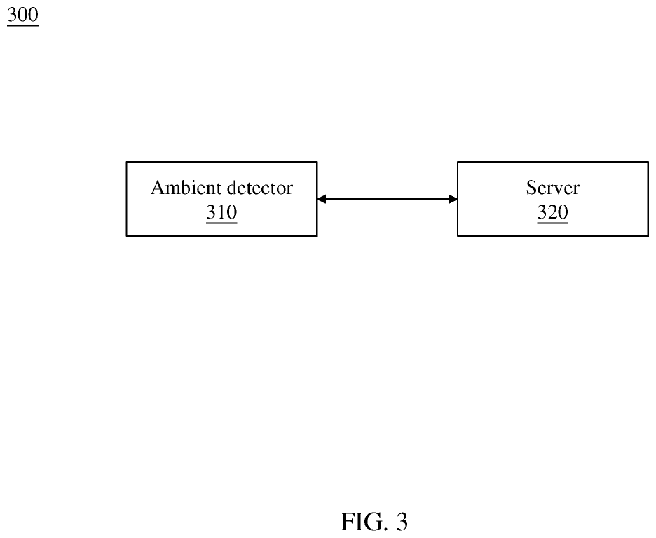

FIG. 3 is a schematic diagram of a system 300 for real-time virus infection risk monitoring in accordance with some embodiments of the present disclosure. As shown in FIG. 3, the system 300 includes an ambient detector 310 and a server 320. The ambient detector 310 may be communicatively connected with the server 320 through the Internet and/or a local area network (e.g., Wi-Fi or Bluetooth).

The ambient detector 310 may be similar to the device 100 in FIG. 1; the difference is that the ambient detector 310 is not responsible for computations on detected ambient values and reference values and determination of virus infection risk. The ambient detector 310 includes a CO₂ sensor, a particulate matter sensor, a temperature sensor, and a humidity sensor for respectively detecting an ambient CO₂ concentration, an ambient particulate matter concentration, an ambient temperature, and an ambient humidity in an environment, and includes a wireless transceiver for sending the ambient CO₂ concentration and the ambient particulate matter concentration to the server 320.

The server 320 may be a local server or a remote server (e.g., a cloud server). After receiving ambient the CO₂ concentration and the ambient particulate matter concentration, the ambient temperature, and the ambient humidity from the ambient detector, the server 320 computes a first difference between the ambient CO₂ concentration and a reference CO₂ concentration, computes a second difference between the ambient particulate matter concentration and a reference particulate matter concentration, and then determines whether the environment has a high infection risk of a specific virus. The server 320 determines that the environment has a high infection risk of a specific virus if the ambient temperature is in a temperature range for transmission of the specific virus, the ambient humidity is in a humidity range for transmission of the specific virus and a condition is met. The condition may be one of the following: the first difference is greater than a first CO₂ concentration difference threshold, and the second difference is greater than a first particulate matter concentration difference threshold; the first difference is greater than the first CO₂ concentration difference threshold, and the second difference is greater than a second particulate matter concentration difference threshold and less than or equal to the first particulate matter concentration difference threshold; or the first difference is greater than a second CO₂ concentration difference threshold and less than or equal to the first CO₂ concentration difference threshold, and the second difference is greater than the first particulate matter concentration difference threshold. After determining that the environment has a high infection risk of a specific virus, the server 320 may send a report to the ambient detector 310 to inform the ambient detector 310 that the environment has the high infection risk of the specific value.

In some embodiments, the ambient detector 310 adjusts the ambient CO₂ concentration, the ambient particulate matter concentration, the ambient temperature, and the ambient humidity according to the current virus or disease information from the server 320 (or another external device) for accurate and instant infection risk determination of a specific virus that is, for example, pandemic around the world or prevalent in an area where the ambient detector 310 is located.

Although particular embodiments of the present disclosure have been described in detail for purposes of illustration, various modifications and enhancements may be made without departing from the spirit and scope of the present disclosure. Accordingly, the present disclosure is not to be limited except as by the appended claims.

What is claimed is:

1. A method for real-time virus infection risk monitoring, the method comprising:
utilizing a CO₂ sensor, a particulate matter sensor, a temperature sensor, and a humidity sensor to respectively detect an ambient CO₂ concentration, an ambient particulate matter concentration, an ambient temperature, and an ambient humidity in a first environment;
computing, by a processor, a first difference between the ambient CO₂ concentration and a reference CO₂ concentration;
computing, by the processor, a second difference between the ambient particulate matter concentration and a reference particulate matter concentration;
determining, by the processor, that the first environment has a high infection risk of a specific virus in response to the ambient temperature being in a temperature range for transmission of the specific virus, the ambient humidity being in a humidity range for transmission of the specific virus, and one of the following conditions being met:
the first difference is greater than a first CO₂ concentration difference threshold, and the second difference is greater than a first particulate matter concentration difference threshold;
the first difference is greater than the first CO₂ concentration difference threshold, and the second difference is greater than a second particulate matter concentration difference threshold and less than or equal to the first particulate matter concentration difference threshold; or
the first difference is greater than a second CO₂ concentration difference threshold and less than or equal to the first CO₂ concentration difference threshold, and the second difference is greater than the first particulate matter concentration difference threshold.

2. The method as claimed in claim 1, further comprising:
controlling, by the processor, an electronic output component to output an indication for an infection risk warning of the specific virus in response to determining that the first environment has the high infection risk of the specific virus.

3. The method as claimed in claim 2, wherein controlling the electronic output component to output the indication is controlling a display to show a health alert message for the infection risk warning of the specific virus.

4. The method as claimed in claim 1, further comprising:
sending, from the processor, a command to a heating, ventilation, and air conditioning (HVAC) system in the first environment to work for adjusting the ambient CO₂ concentration, the ambient particulate matter concentration, the ambient temperature, or the ambient humidity.

5. The method as claimed in claim 1, wherein the ambient temperature is less than 30° C., the ambient humidity is less than 45% or greater than 90%, and the specific virus is coronavirus.

6. The method as claimed in claim 1, wherein the ambient temperature is less than 22° C., the ambient humidity is less than 50%, and the specific virus is influenza.

7. The method as claimed in claim 1, wherein the reference particulate matter concentration and the ambient particulate matter concentration are PM2.5 concentrations or PM10 concentrations.

8. The method as claimed in claim 1, wherein the first CO₂ concentration difference threshold is 1200 ppm, the second CO₂ concentration difference threshold is 400 ppm, the first particulate matter concentration difference threshold is 40 μg/m³, and the second particulate matter concentration difference threshold is 20 μg/m³.

9. The method as claimed in claim 1, wherein the reference CO₂ concentration and the reference particulate matter concentration are provided from a local database or a cloud database.

10. The method as claimed in claim 1, wherein the reference CO₂ concentration and the reference particulate matter concentration are detected in a second environment different from the first environment and respectively by the CO₂ sensor and the particulate matter sensor.

11. The method as claimed in claim 10, wherein the first environment and the second environment correspond to an identical space and different detection times.

12. The method as claimed in claim 10, wherein the first environment corresponds to an indoor space, and wherein the second environment corresponds to an outdoor space neighboring the indoor space.

13. The method as claimed in claim 1, further comprising:
sending, from the processor, an indication with a location of the first environment to an external device for an infection risk warning of the specific virus for the first environment after determining that the first environment has the high infection risk of the specific virus.

14. The method as claimed in claim 1, further comprising:
adjusting, by the processor, the reference CO₂ concentration, the reference particulate matter concentration, the temperature range, and the humidity range of the specific virus according to current virus of disease information.

15. A device for real-time virus infection risk monitoring, the device comprising:
a CO₂ sensor configured to detect an ambient CO₂ concentration in an environment;
a particulate matter sensor configured to detect an ambient particulate matter concentration in the environment;
a temperature sensor configured to detect an ambient temperature in the environment;
a humidity sensor configured to detect an ambient humidity in the environment; and
a processor configured to:
compute a first difference between the ambient CO₂ concentration and a reference CO₂ concentration;
compute a second difference between the ambient particulate matter concentration and a reference particulate matter concentration; and
determine that the environment has a high infection risk of a specific virus in response to the ambient temperature being in a temperature range for transmission of the specific virus, the ambient humidity being in a humidity range for transmission of the specific virus, and one of the following conditions being met:
the first difference is greater than a first CO₂ concentration difference threshold, and the second difference is greater than a first particulate matter concentration difference threshold;
the first difference is greater than the first CO₂ concentration difference threshold, and the second difference is greater than a second particulate matter concentration difference threshold and less than or equal to the first particulate matter concentration difference threshold; or
the first difference is greater than a second CO₂ concentration difference threshold and less than or equal to the first CO₂ concentration difference threshold, and the second difference is greater than the first particulate matter concentration difference threshold.

16. The device as claimed in claim 15, further comprising:
an electronic output component configured to output an indication for an infection risk warning of the specific virus in response to the environment being determined to have the high infection risk of the specific virus.

17. The device as claimed in claim 15, wherein the processor is further configured to send an indication with a location of the environment to an external device through a wireless transceiver for an infection risk warning of the specific virus for the environment after the environment is determined to have the high infection risk of the specific virus.

18. The device as claimed in claim 15, wherein the processor is further configured to send an indication with a location of the environment to an external device for an infection risk warning of the specific virus for the environment after determining that the environment has the high infection risk of the specific virus.

19. The device as claimed in claim 15, wherein the processor is further configured to adjust the reference CO₂ concentration, the reference particulate matter concentration, the temperature range, and the humidity range of the specific virus according to current virus of disease information.

20. A system for real-time virus infection risk monitoring, the system comprising:
an ambient detector, comprising:
a CO₂ sensor configured to detect an ambient CO₂ concentration in an environment;
a particulate matter sensor configured to detect an ambient particulate matter concentration in the environment;
a temperature sensor configured to detect an ambient temperature in the environment;
a humidity sensor configured to detect an ambient humidity in the environment; and
a wireless transceiver configured to send the ambient CO₂ concentration, the ambient particulate matter concentration, the ambient temperature, and the ambient humidity;
a server communicatively connected to the ambient detector, the server configured to:
receive the ambient CO₂ concentration, the ambient particulate matter concentration, the ambient temperature, and the ambient humidity from the ambient detector;
compute a first difference between the ambient CO₂ concentration and a reference CO₂ concentration;
compute a second difference between the ambient particulate matter concentration and a reference particulate matter concentration; and
determine that the environment has a high infection risk of a specific virus in response to the ambient temperature being in a temperature range for transmission of the specific virus, the ambient humidity being in a humidity range for transmission of the specific virus, and one of the following conditions being met:
the first difference is greater than a first CO₂ concentration difference threshold, and the second difference is greater than a first particulate matter concentration difference threshold;
the first difference is greater than the first CO₂ concentration difference threshold, and the second difference is greater than a second particulate matter concentration difference threshold and less than or equal to the first particulate matter concentration difference threshold; or
the first difference is greater than a second CO₂ concentration difference threshold and less than or equal to the first CO₂ concentration difference threshold, and the second difference is greater than the first particulate matter concentration difference threshold; and
send a report to the ambient detector to inform the ambient detector that the environment has the high infection risk of the specific virus.
